(19) United States
(12) Patent Application Publication
(10) Pub. No.: US 2022/0265571 A1
(43) Pub. Date: Aug. 25, 2022

(54) COMPOSITION OF CANNABIDIOL FOR USE IN THE TREATMENT AND PREVENTION OF BRONCHIOLITIS OBLITERANS

(71) Applicant: MOR RESEARCH APPLICATIONS LTD., Tel Aviv (IL)

(72) Inventor: Moshe YESHURUN, Elqana (IL)

(21) Appl. No.: 17/623,126

(22) PCT Filed: Jun. 28, 2020
(86) PCT No.: PCT/IL2020/050719

§ 371 Date: Dec. 27, 2021

Related U.S. Application Data
(60) Provisional application No. 62/867,287, filed on Jun. 27, 2019.

(51) Int. Cl. A61K 31/05

(52) U.S. Cl. CPC A61K 31/05

(57) ABSTRACT

The present invention is directed to a pharmaceutical composition comprising Cannabidiol and a method of use thereof, i.e., for treating or preventing bronchiolitis obliterans in a subject.

### FIELD OF INVENTION

The present invention, in some embodiments thereof, is in the field of cannabidiols, and is directed to methods of using the same such as for treating bronchiolitis obliterans disease.

### BACKGROUND OF THE INVENTION

Cannabidiol (CBD) is a major non-psychotropic constituent of Cannabis. CBD has anti-convulsive, anti-anxiety, anti-psychotic, anti-nausea and anti-inflammatory and immunomodulatory properties. CBD binds to cannabinoid and non-cannabinoid receptors and its mechanism of action is not fully known.

The chemical nomenclature of CBD differs from that of Tetrahydrocannabinol (THC). While the latter has a pyran ring, which determines its numbering, CBD has no heterocyclic ring and its numbering stems from that of the terpene ring. The chemistry of CBD has been well explored over the last 50 years. In view of the various, potentially therapeutic, effects caused by CBD, it seems plausible that novel synthetic approaches will still be developed in the future to lead to new types of derivatives.

Bronchiolitis obliterans, commonly nicknamed as “popcorn lung” is a pulmonary disease characterized by fixed airway obstruction. Bronchiolitis obliterans can be induced by numerous factors, such as collagen vascular disease, rejection of transplanted lungs, viral infection, Stevens-Johnson syndrome, and others. In addition, bronchiolitis obliterans can be idiopathic. The disease is considered to be irreversible, with lung transplant being offered, yet often results with poor outcomes, with most people dying in a period of months to years.

A method of treating or preventing bronchiolitis obliterans is therefore greatly needed.

### SUMMARY OF THE INVENTION

According to one aspect, there is provided a method for treating or preventing bronchiolitis obliterans in a subject comprising administering to the subject a therapeutically effective amount of Cannabidiol (CBD), thereby treating or preventing bronchiolitis obliterans in the subject.

In some embodiments, a pharmaceutical composition comprising CBD for use in the treatment of bronchiolitis obliterans, is provided.

In some embodiments, the CBD is administered in combination with at least one immunosuppressant.

In some embodiments, the at least one immunosuppressant is cyclosporine.

In some embodiments, the CBD and the at least one immunosuppressant are administered simultaneously or sequentially.

In some embodiments, the administering comprises orally administering, intranasally administering, or both.

In some embodiments, the administering is by a vaporizer or a humidifier.

In some embodiments, the subject is at increased risk of developing bronchiolitis obliterans, compared to control.

In some embodiments, the method further comprises a preliminary step of determining the subject is at increased risk of developing bronchiolitis obliterans, compared to control.

In some embodiments, the subject is afflicted with an inflammatory pulmonary disease.

In some embodiments, the inflammatory pulmonary disease comprises one or more diseases or conditions selected from the group consisting of: pneumocystis pneumonia, bronchopulmonary dysplasia, drug reaction, and exposure to toxic fumes.

In some embodiments, the toxic fumes are selected from the group consisting of: diacetyl, sulfur dioxide, nitrogen dioxide, ammonia, chlorine, thionyl chloride, methyl isocyanate, hydrogen fluoride, hydrogen bromide, hydrogen chloride, hydrogen sulfide, phosgene, polyamide-amine dye, mustard gas, and ozone.

In some embodiments, the CBD is administered in a daily dose of 5 mg to 1,200 mg.

In some embodiments, the administering is administering 1-5 times per day.

In some embodiments, the CBD is in a pharmaceutical composition with an acceptable carrier.

In some embodiments, the pharmaceutical composition is an inhalation composition.

In some embodiments, the treatment comprises reducing the risk of developing bronchiolitis obliterans, prevention of bronchiolitis obliterans, or both.

In some embodiments, the pharmaceutical composition further comprises an immunosuppressant.

In some embodiments, the immunosuppressant is ciclosporin.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

According to some embodiments, a method for treating or preventing bronchiolitis obliterans in a subject comprising administering to the subject a therapeutically effective amount of Cannabidiol (CBD), thereby treating or preventing bronchiolitis obliterans in the subject, is provided.

According to the method of the invention, in some embodiments thereof, CBD is administered in a daily dose of at least 5 mg, at least 15 mg, at least 50 mg, at least 100 mg, at least 200 mg, at least 300 mg, at least 400 mg, at least 500 mg, or at least 600 mg, at least 750 mg, at least 1,000 mg, or at least 1200 mg, or any value and range therebetween. Each possibility represents a separate embodiment of the invention. In some embodiments, CBD is administered in a daily dose of 5-50 mg, 10-100 mg, 10-250 mg, 50-350 mg, 100-700 mg, 5-500 mg, 220-450 mg, 70-400 mg, 1-350 mg, 30-490 mg, 50-550 mg, 150-850 mg, 250-1,000 mg, or 400-1,250 mg. Each possibility represents a separate embodiment of the invention.

In some embodiments, CBD is administered in combination with at least one immunosuppressant. In some embodiments, at least one comprises at least 2, at least 3, at least 4, at least 5, at least 7, at least 8, or at least 10, or any value and range therebetween. Each possibility represents a separate embodiment of the invention. In some embodiments, at least one comprises 1-5, 2-7, 3-5, 1-8, 4-9, or 3-10. Each possibility represents a separate embodiment of the invention.

As used herein, the term “immunosuppressant” refers to a compound that reduces, inhibits, or suppresses a body's immune system.

For clarity, a suppressed immune system may be a desired outcome or a required outcome, such as for example, prior to or following an organ transplantation. A suppressed immune system in some cases may not induce a transplant rejection reaction, may be less robust, and may not recognize a compatible transplant as “non-self” or foreign. Also, a suppressed immune system may not recognize cells of the host, or molecules produced or secreted therefrom, as non-self or foreign.

In some embodiments, CBD and the at least one immunosuppressant are administered concomitantly. In some embodiments, CBD and the at least one immunosuppressant are administered consecutively. In some embodiments, CBD and the at least one immunosuppressant are administered sequentially. In some embodiments, CBD and the at least one immunosuppressant are administered simultaneously.

In some embodiments, the method comprises administering CBD either alone or in combination with at least one immunosuppressant at least 1 time, at least 2 times, at least 3 times, at least 4 times, at least 5 times, at least 7 times, or at least 10 times per day, or any value and range therebetween. Each possibility represents a separate embodiment of the invention. In some embodiments, the method comprises administering CBD either alone or in combination with at least one immunosuppressant 1-2 times per day, 1-3 times per day, 1-4 times per day, 1-5 times per day, 1-7 times per day, 2-3 times per day, 2-4 times per day, 2-5 times per day, 3-4 times per day, 3-5 times per day, or 5-7 times per day. Each possibility represents a separate embodiment of the invention.

In some embodiments, CBD and the at least one immunosuppressant are formulated in separate pharmaceutical compositions and are administered as disclosed herein. In some embodiments, CBD and the at least one immunosuppressant formulated in a single pharmaceutical composition.

Immunosuppressants classes, types, and administration dose, would be apparent to one of skill in the art.

In some embodiments, the immunosuppressant is selected from: a glucocorticosteroid, a cytostatic (i.e., inhibit cell division), an antibody, an immunophilin-targeting drug, or others.

In some embodiments, an immunosuppressing glucocorticosteroid is selected from: prednisone, dexamethasone, and hydrocortisone.

In some embodiments, an immunosuppressing cytostatic compound is selected from: a nitrogen mustard (e.g., cyclophosphamide), nitrosoureas, platinum, a folic acid analogue (e.g., methotrexate), a purine analogue (e.g., azathioprine, mercaptopurine), a pyrimidine analogue (e.g., fluorouracil), a protein synthesis inhibitor, a cytotoxic antibiotic compound (e.g., dactinomycin, anthracyclines, mitomycin C, and bleomycin, mithramycin).

In some embodiments, an immunosuppressing antibody is selected from: anti-CD-25 (i.e., IL-2 receptor) antibody, anti-CD-3 antibody. Non-limiting examples of immunosuppressing antibodies include, but are not limited to, Muromonab-CD3, Basiliximab (Simulect), and Daclizumab (Zenapax).

In some embodiments, an immunosuppressing immunophilin-targeting drug (i.e., a drug acting on immunophilin) is selected from: Ciclosporin, Tacrolimus, Sirolimus, Everolimus.

In some embodiments, other immunosuppressing compounds can be selected from: interferon (e.g., INFγ, and INFβ), Mycophenolate, fingolimod, a TNFα-binder (e.g., Infliximab, Adalimumab, and Etanercept), and opioids

In some embodiments, the immunosuppressant is cyclosporine.

In some embodiments, administering is orally administering. In some embodiments administering is intranasally administering. In some embodiments, administering is orally administering and intranasally administering.

In some embodiments, administering comprises the use of a vaporizer. In some embodiments, administering comprises the use of a humidifier.

As used herein, a vaporizer or a humidifier may receive a composition in a liquid form. In some embodiments, a vaporizer or a humidifier may convert the composition into a vapor and/or an aerosol to be inhaled or otherwise received by a subject.

The structure and operation of a vaporizer will be apparent to one of ordinary skill in the art. However, in the interest of clarity, the elements of a vaporizer are briefly discussed. A vaporizer may include a cartridge, an atomizer, and a battery. The cartridge may include a reservoir to hold a liquid to be vaporized. The atomizer may include a heating element to convert the liquid into a vapor and/or aerosol. Optimally, the atomizer can vaporize the liquid without initiating combustion. The battery may have an electrical charge to power the atomizer and other accessories, for example, an indicator light that illuminates while the electronic cigarette is operating.

Vaporization provides many advantages over traditional combustion based methods of administering cannabinoids. For example, since a vaporizer can convert a composition into a vapor, the levels of each ingredient in the composition, including the cannabinoid, are controllable. Also, since vaporization does not involve combustion, a user may inhale or otherwise receive a bio-active ingredient, for example via inhalation, without being required to receive high levels of tar and various toxins associated with smoking. Vaporization also benefits from advantages such as rapid intake, direct delivery to the bloodstream, enhanced control of over- and under-dosage, and avoidance of respiratory disadvantages associated with combustion-based smoking. Vaporization may occur at approximately 180-190° C., which may significantly reduce pyrolytic smoke compound generation. Additionally, vaporization may occur below the typical point of combustion where smoke and associated toxins are generated (e.g., 230° C.).

In some embodiments, the method further comprises a preliminary step of determining the subject is at increased risk of developing bronchiolitis obliterans. As used herein, the term “preliminary” refers to that the determining of the subject being at increased risk of developing bronchiolitis obliterans precedes the treating of the subject.

Methods of determining bronchiolitis obliterans in a subject or increased risk of developing the same, would be apparent to one of ordinary skill in the art. Non-limiting examples for such methods include, but are not limited to, lung diffusing capacity tests (DLCO), chest X-rays, high-resolution CT (HRCT), lung biopsy, lung volume tests, and spirometry.

In some embodiments, a subject at increased risk of developing bronchiolitis obliterans has greater probability of developing bronchiolitis obliterans, compared to control. In some embodiments, a subject at increased risk of developing bronchiolitis obliterans is more susceptible to develop bronchiolitis obliterans compared to control. In some embodiments, a subject at increased risk of developing Bronchiolitis obliterans has poor prognosis compared to control.

The phrases “increased risk of developing bronchiolitis obliterans”, “greater probability of developing bronchiolitis obliterans”, and “more susceptible to develop bronchiolitis obliterans” are used herein interchangeably.

In some embodiments, the control is a healthy subject. In some embodiments, the control is a subject having low risk or no risk of developing bronchiolitis obliterans. In some embodiments, the control is a subject having a low probability of developing bronchiolitis obliterans. In some embodiments, the control is a subject unsusceptible to develop bronchiolitis obliterans.

In some embodiments, a subject at risk of developing bronchiolitis obliterans is at least 5%, at least 10%, at least 20%, at least 35%, at least 50%, at least 100%, at least 200%, at least 350%, at least 500%, at least 750%, or at least 1,000% more susceptible of developing bronchiolitis obliterans, compared to control, or any value and range therebetween. Each possibility represents a separate embodiment of the invention. In some embodiments, a subject at risk of developing bronchiolitis obliterans is 5-100%, 10-200%, 20-500%, 35-850%, 50-450%, 100-550%, 200-900%, 350-875%, 500-1,150%, 750-1,200%, or 250-1,000% more susceptible of developing bronchiolitis obliterans, compared to control. Each possibility represents a separate embodiment of the invention.

In some embodiments, a control has a probability of 1% at most, 2% at most, 3% at most, 5% at most, 6% at most, 7% at most, 8% at most, 9% at most, 10% at most of developing bronchiolitis obliterans, or any value and range therebetween. Each possibility represents a separate embodiment of the invention. Each possibility represents a separate embodiment of the invention. In some embodiments, a control has a probability of 1-7%, 1-8%, 1-9%, 1-10%, 2-8%, 2-9%, 2-12%, 3-9%, 4-13%, 5-10%, 3-7%, or 5-15% of developing Bronchiolitis obliterans.

In some embodiments, the subject is afflicted with an inflammatory pulmonary disease. In some embodiments, an inflammatory pulmonary disease comprises one or more diseases or conditions selected from: hypersensitivity lung disease, hypersensitivity pneumonitis, delayed-type hypersensitivity, interstitial lung disease (ILD), idiopathic pulmonary fibrosis, ILD associated with rheumatoid arthritis, pneumocystis pneumonia, bronchopulmonary dysplasia, asthma, allergic asthma, emphysema, chronic obstructive pulmonary disease or disorder, sarcoidosis and bronchitis.

In some embodiments, the subject is undergoing or undergone a lung transplantation.

In some embodiments, the subject is undergoing or undergone a bone marrow transplantation.

In some embodiments, the subject is afflicted with or suffering from a long-term oxidative stress.

In some embodiments, the subject is afflicted with or suffering from high malondialdehyde (e.g., a biomarker of oxidative stress, known to formed when fats are oxidized). In some embodiments, the subject is afflicted with or suffering from increased lipid peroxidation.

In some embodiments, the subject is afflicted with or suffering from environmental pollutants. In some embodiments, the subject is afflicted with or suffering from exposure to toxic fumes. In some embodiments, the subject is afflicted with or suffering from a drug reaction.

In some embodiments, types of toxic fumes are selected from: diacetyl, sulfur dioxide, nitrogen dioxide, ammonia, chlorine, thionyl chloride, methyl isocyanate, hydrogen fluoride, hydrogen bromide, hydrogen chloride, hydrogen sulfide, phosgene, polyamide-amine dye, mustard gas, and ozone. In some embodiments, the aforementioned toxic fumes increase the risk of Bronchiolitis obliterans development in a subject. In some embodiments, active and/or passive exposure to cigarettes' smoke increase the risk of Bronchiolitis obliterans development in a subject.

In some embodiments, the subject is a smoker or an ex-smoker.

In another embodiment, the subject is a mammal. In another embodiment, the subject is a lab animal. In another embodiment, the subject is a pet. In another embodiment, the subject is a rodent. In another embodiment, the subject is a farm animal. In another embodiment, the subject is a human subject.

As used herein, the term “subject” refers to any subject for whom therapy is desired, for example, a human.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “treating” comprises slowing down the progression of a disease progression. In some embodiments, treating comprises preventing the disease. In some embodiments, treating comprises reducing the risk of developing a disease. In some embodiments, treating comprises reducing the rate of disease deterioration.

### Cannabidiol

The method as described herein, in some embodiments thereof, comprises administration of Cannabidiol (CBD).

As used herein, the term “CBD” comprises CBD, any functional derivative thereof, or any combination thereof. According to some embodiments, CBD or any functional derivative thereof refers to compounds and/or compositions comprising the same that are substantially and/or essentially devoid of Tetrahydrocannabinol (THC). In one embodiment, a composition comprising CBD as described herein is substantially and/or essentially devoid of THC. As used herein, CBD or any functional derivative thereof, according to some embodiments, refers to compounds and/or compositions comprising the same in an amount of at least 80%, at least 90% CBD, at least 92% CBD, at least 95% CBD, at least 97%, or at least 99% CBD or any functional derivative thereof, or any value and range therebetween. Each possibility represents a separate embodiment of the invention. In some embodiments, CBD or any functional derivative thereof, according to some embodiments, refers to compounds and/or compositions comprising 80-90%, 85-95%, 92-99%, or 93-100%. Each possibility represents a separate embodiment of the invention.

In one embodiment, substantially and/or essentially devoid of THC refers to the % by weigh or weight/weight. In some embodiments, substantially and/or essentially devoid of THC is less than 10% by weight, less than 7% by weight, less than 5% by weight, less than 3% by weight, less than 1% by weight, less than 0.5% by weight, less than 0.3% by weight, or less than 0.1% by weight or weight/weight THC, or any value and range therebetween. Each possibility represents a separate embodiment of the invention. In some embodiments, substantially and/or essentially devoid of THC is 0.1-0.5% by weight, 0.3-3% by weight, 1-5% by weight, 3-7% by weight, 6-10% by weight. Each possibility represents a separate embodiment of the invention.

In the methods and compositions described herein, according to some embodiments thereof, the phrase “purified or substantially purified” refers to greater than 80% w/w, 85% w/w, 90%, w/w 95% w/w or 97% w/w cannabidiol, or a functional variant thereof, CBD, also termed 2-[(6R)-3-Methyl-6-prop-1-en-2-yl-1cyclohex-2-envyl]-5pentylbenzene-1,3-diol, has the molecular formula of C₂₁H₃₀O₂.

The chemical structure of CBD is shown hereinbelow in Formula I:

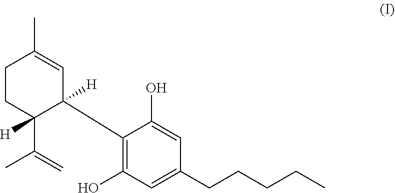 [SMILES: C=C(C)[C@@H]1CCC(C)=C[C@H]1c1c(O)cc(CCCCC)cc1O]

Cannabidiol is insoluble in water but soluble in organic solvents, such as oil. Accordingly, CBD can be formulated for use in the described method through use of any organic solvent known to the pharmaceutical arts, including, but not limited to edible oils. When formulated for oral administration, any edible oil can be used in the CBD formulation, including olive oil.

In some embodiments, a CBD derivative, comprises a metabolite of CBD such as but not limited to: (−)-7-hydroxy-CBD and (−)-CBD-7-oic acid and their dimethylheptyl (DMH) homologs, as well as of the corresponding compounds in the enantiomeric (+)-CBD series. A CBD derivative is characterized, in some embodiments, by a structure wherein at least one of the hydroxyl substituent groups is converted to a stable form thereof. In one embodiment, a CBD derivative is cannabinol comprising a quinone ring. In one embodiment, a CBD derivative is an endocannabinoid derivative. In some embodiments, a CBD derivative is described in Frank D King; G Lawton; A W Oxford Progress in medicinal chemistry. Vol. 44. Pages 207-331, Elsevier Science, 2006 ISBN: 0080462103 9780080462103 which is hereby incorporated by reference in its entirety.

### Composition

According to some embodiments, a pharmaceutical composition comprising CBD with an acceptable carrier, is provided. In some embodiments, the pharmaceutical composition further comprises at least one immunosuppressant.

Non-limiting examples of carriers include, but are not limited to, terpenes derived from Cannabis, or total terpene extract from Cannabis plants, terpenes from coffee or cocoa, mint-extract, eucalyptus-extract, citrus-extract, tobacco-extract, anis-extract, propylene glycol, ethanol, water, sodium chloride, peppermint oil, any vegetable oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, phenyl acetic acid and its derivatives, diclofenac, nepafenac, bromfenac, phytol, terpineol, terpinen-4-ol, thymol, and thymoquinone.

In some embodiments, the pharmaceutical composition is for use in the treatment or prevention of bronchiolitis obliterans.

As used herein, the term “prevention” comprises reducing the risk of developing bronchiolitis obliterans, as elaborated hereinabove.

In some embodiments, the disclosed pharmaceutical composition is an inhalation composition.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising” indicate the inclusion of any recited integer or group of integers but not the exclusion of any other integer or group of integers.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises” “comprising”, and “having” are/is interchangeable with “consisting”.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for treating or preventing bronchiolitis obliterans in a subject comprising administering to said subject a therapeutically effective amount of Cannabidiol (CBD), thereby treating or preventing bronchiolitis obliterans in the subject.

2. The method of claim 1, wherein said CBD is administered in combination with at least one immunosuppressant.

3. The method of claim 2, wherein said at least one immunosuppressant is cyclosporine.

4. The method of claim 2, wherein said CBD and said at least one immunosuppressant are administered simultaneously or sequentially.

5. The method of claim 1, wherein said administering comprises orally administering, intranasally administering, or both.

6. The method of claim 1, wherein said administering is by a vaporizer or a humidifier.

7. The method of claim 1, wherein said subject is at increased risk of developing bronchiolitis obliterans, compared to control.

8. The method of claim 1, further comprising a preliminary step of determining said subject is at increased risk of developing bronchiolitis obliterans, compared to control.

9. The method of claim 1, wherein said subject is afflicted with an inflammatory pulmonary disease.

10. The method of claim 9, wherein said inflammatory pulmonary disease comprises one or more diseases or conditions selected from the group consisting of: pneumocystis pneumonia, bronchopulmonary dysplasia, drug reaction, and exposure to toxic fumes.

11. The method of claim 10, wherein said toxic fumes are selected from the group consisting of: diacetyl, sulfur dioxide, nitrogen dioxide, ammonia, chlorine, thionyl chloride, methyl isocyanate, hydrogen fluoride, hydrogen bromide, hydrogen chloride, hydrogen sulfide, phosgene, polyamide-amine dye, mustard gas, and ozone.

12. The method of claim 1, wherein said CBD is administered in a daily dose of 5 mg to 1,200 mg.

13. The method of claim 1, wherein said administering is administering 1-5 times per day.

14. The method of claim 1, wherein said CBD is in a pharmaceutical composition with an acceptable carrier.

15. (canceled)

16. (canceled)

17. (canceled)

18. (canceled)

19. (canceled)
